(19) United States
(12) Patent Application Publication
(10) Pub. No.: US 2013/0338677 A1
(43) Pub. Date: Dec. 19, 2013

(54) INSERTION ELEMENT AND INSERTION DEVICE

(71) Applicant: BIOTRONIK AG, Buelach (CH)

(72) Inventors: Alwin Schwitzer, Buelach (CH); Amir Fargahl, Buelach (CH)

(73) Assignee: Biotronik AG, Buelach (CH)

(21) Appl. No.: 13/837,273

(22) Filed: Mar. 15, 2013

Related U.S. Application Data
(63) Continuation-in-part of application No. 13/709,805, filed on Dec. 10, 2012, now abandoned.
(60) Provisional application No. 61/660,831, filed on Jun. 18, 2012.

(51) Int. Cl. A61F 2/00

(52) U.S. Cl. CPC A61F 2/0095
USPC 606/108

(57) ABSTRACT

One embodiment of the invention relates to an insertion element which is designed to cooperate with an insertion device for inserting a medical implant into a human or animal body. The insertion element comprises a sleeve which, in the circumferential direction, has at least two types of circumferential segments of different resilience.

The invention further includes an embodiment relating to an insertion device for inserting a medical implant into a human or animal body, comprising at least one outer insertion element wherein the outer insertion element is expand able, at least in regions, when ready for use, wherein the outer insertion element comprises a sleeve which, in the circumferential direction, has at least two types of circumferential segments of different resilience.

### CROSS REFERENCE

The present application claims the benefit of and priority on co-pending U.S. Provisional Application No. 61/660,831 filed on Jun. 18, 2012; which application is incorporated herein by reference. The present application is a continuation in part of co-pending U.S. application Ser. No. 13/709,805 filed Dec. 10, 2012 which application is likewise incorporated herein by reference.

### TECHNICAL FIELD

One aspect of the invention relates to an insertion element and to an insertion device configured for use in implanting medical devices in a person.

### BACKGROUND

Implants that are introduced into an animal and/or human body either permanently or at least for a relatively long period of time to carry out replacement functions are often used in the field of medicine. For example, these include cardiac pacemakers, brain pacemakers for Parkinson's patients, cardiac implants, cochlear implants, retinal implants, dental implants, joint replacement implants, vessel prostheses or stents.

Before being inserted into the body, implants are connected to catheters, with the aid of which they can be placed accurately at the site of use and can be released in a defined manner. For introduction into the animal and/or human body, a tubular insertion element is used for this purpose, through which the implant is slid by means of the insertion device. So as to prevent the stressing of the vessel when the implant is introduced, an insertion element is known from US 2010/0094392 A1, which is only expanded to the necessary larger diameter once the implant has been passed through. The insertion element consists of two to three coaxially arranged layers, wherein the outer layer is provided with longitudinal slits, which enables the enlargement of the diameter.

One object of the invention is to disclose an insertion element that is simplified in terms of its production and that enables improved flexibility of the insertion element.

A further object is the provision of a corresponding insertion device. Other objects are also addressed and achieved through the invention.

### SUMMARY

The objects are achieved in accordance with the invention by the features of the independent claims. Advantageous embodiments and advantages of the invention will emerge from the further claims and from the description.

An insertion element, which in some embodiments may comprise an introducer, is provided through the invention, which is designed to cooperate with an insertion device for inserting a medical implant into a human or animal body. The insertion element has a sleeve, which, in the circumferential direction, has at least two types of circumferential segments of different resilience.

### DESCRIPTION OF THE DRAWINGS

The invention is explained in greater detail herein after by way of example on the basis of exemplary embodiments illustrated in drawings, in which, schematically:

FIGS. 1A and 1B show a side view (FIG. 1A) and a cross section (FIG. 1B) of a first exemplary embodiment of an insertion element according to the invention; and

FIGS. 2A and 2B show a side view (FIG. 2A) and a cross section (FIG. 2B) of a further exemplary embodiment of an insertion element according to the invention;

FIGS. 3A and 3B show a side view (FIG. 3A) and a cross section (FIG. 3B) of a further exemplary embodiment of an insertion element according to the invention;

FIG. 4 shows a side view of an exemplary embodiment of an insertion device according to the invention;

FIGS. 5A and 5B show a side view of an insertion situation of an insertion device according to an embodiment of the invention with a partially released implant (FIG. 5A) and with an implant re-sheathed with the insertion element slid over (FIG. 5B).

FIGS. 6A and 6B show a side view (FIG. 6A) and a cross section (FIG. 6B) of a further example embodiment of an insertion element according to the invention.

### DETAILED DESCRIPTION

An insertion element is advantageously created with a resiliently expand able diameter, in particular a mechanically stable tube, of which the diameter enlarges under the action of a force and returns to the original diameter when the effective force is removed, similarly to an elastic spring. Before discussing various embodiments illustrated in the Figures, further discussion of some features will be helpful.

A circumferential segment of the sleeve is to be understood to mean a region of the sleeve that can be formed as a narrow or wide strip to complete a lateral surface of the sleeve. A sleeve is understood to mean a generally tubular shaped body that is formed continuously, in particular in a materially bonded manner, from one or more material components, in other words the at least two types of circumferential segments are formed integrally with one another. A particularly stable and reliable construction of the sleeve can thus be achieved. In this case, “integral” is to be understood to mean that the circumferential segments of the at least two types can only be separated from one another with a loss of function of at least one of the circumferential segments.

Different resilience is to be understood to mean that the resilience, in particular the modulus of elasticity, of the material of the first type of circumferential segment differs considerably from the material of the second type of circumferential segment under otherwise identical conditions, in particular at least by a factor of 2. In particular, the ductility, in particular the elongation at yield, of the materials of the first type of circumferential segment and the second type of circumferential segment differ considerably under otherwise identical conditions.

One of the types of circumferential segments is formed resiliently. Resilient is to be understood in particular to mean that the respective circumferential segment reversibly re-assumes its original dimensions after a stretching process by which the diameter of the insertion element is enlarged.

In accordance with an advantageous embodiment, one type of the at least two circumferential segments may be much stiffer in the circumferential direction than the other type of the at least two circumferential segments. The insertion element is advantageously mechanically stable on the one hand. The flexibility of the insertion element when ready for use can thus be improved. An undesired kinking of the insertion element with tight radii of curvature, as can occur with an insertion element of the prior art having longitudinal slits, can be reliably avoided. On the other hand, a reversible extension of diameter is enabled, which allows the implant to be passed within the insertion element without difficulty.

An advantageous embodiment may provide a tubular embodiment of the insertion element, wherein the at least two types of circumferential segments may extend or be arranged along a longitudinal extension of the insertion element. The respective circumferential segments may advantageously be formed over the entire length of the insertion element with widths (measured in the circumferential direction) which are constant, based on the circumference, and which can be produced in a particularly simple manner. However, it is also conceivable for the widths of the different and/or identical circumferential segments to vary along the length of the insertion element, such that a length-variable ductility of the insertion element can be provided.

In accordance with an advantageous embodiment, one type of circumferential segment may comprise thermoplastic material. Suitable materials include polyether ether ketone (PEEK), polyimide (PI) polyether ketone ketone (PEKK), polyamides, polyester, polyethylene (PE), polypropylene (PP), polycarbonate (PC). For example, polyamides, for example polyamide 12 (PA-12, consisting of laurolactam or ω-amino dodecanoic acid) or polyester, for example polyethylene terephthalate, are preferably known as PET.

In accordance with an advantageous embodiment, another type of circumferential segment may comprise a thermoplastic from the class of thermoplastic elastomers (TPEs). Thermoplastic elastomers selected from thermoplastic elastomers based on olefin TPE-Os (TPOs), thermoplastic elastomers based on urethane TPE-Us (TPUs), styrene block copolymers TPE-S′ (TPS′), thermoplastic copolyesters TPE-Es or thermoplastic copolyamides TPE-As (TPAs), or combinations thereof are suitable. For example, polyamide elastomers, for example polyether block amide block copolymers (PEBAs), for example PEBAX (trade name of Arkema) or polyester elastomers, for example Hytrel (trade name of Dupont), or combinations thereof are preferred.

The at least first and second types of circumferential segments may advantageously be formed by materials that are compatible with one another. These may easily be joined by simple material bonding at the melting point to form a closed sleeve of the insertion element. In particular, a thermoplastic material for example can be joined with a thermoplastic elastomer, for example polyamide PA-12 can be joined with a polyamide elastomer or polyester can be joined with a polyester elastomer.

In accordance with an advantageous embodiment, the sleeve can be formed with the different circumferential segments by co-extrusion. This is a particularly simple option for producing the insertion element, in which the different, yet compatible, materials of the circumferential segments of the sleeve can be interconnected with simultaneous formation of the sleeve. It is not necessary to join a plurality of concentric material layers to form a sleeve. Nor is there a need for any postprocessing to incorporate openings or slits into the sleeve so that the diameter thereof can expand. The variation in the resilience of the sleeve along the circumference with simultaneously sufficient strength is an intrinsic feature of the sleeve and enables a reversible dilation of the insertion element when the implant is passed through, which allows a rapid and gentle insertion and placement of the implant.

In accordance with an advantageous embodiment, at least one stabilizing element (and in some embodiments more than one stabilizing elements) can be arranged in at least one of the circumferential segments along a longitudinal extension of the insertion element or the sleeve. The at least one stabilizing element is expediently arranged in the region of the sleeve that is the less ductile region and/or in the region of the sleeve having the greater circumferential area (matrix). The stabilizing element may extend in particular continuously in the axial direction of the insertion element or the sleeve, at least over a predefined region (in some embodiments the stabilizing element extends for only a portion of the length of the insertion element or sleeve). The stabilizing element may advantageously be designed as a wire for example or as a wire bundle, as a fiber or fiber bundle, or as a mixture of one or more embodiments. For example, the sleeve may be equipped with one or more stabilizing elements in a first sleeve region proximate to the end that is the distal end when assembled, and may be otherwise free from stabilizing elements or may be free from stabilizing elements in a second sleeve region proximate to the other, proximal end. A reverse arrangement is likewise conceivable.

In accordance with an advantageous embodiment, the mentioned stabilizing element or elements can be molded. The profiled stabilizing elements can be configured so that the inner contact area of the insertion element is minimized. In one embodiments, this is achieved by having an interior surface that has raised portions relative to other portions, with the raised portions extending into the interior farther than non-raised portions to thereby define a reduced area contact area for engaging an article inserted into the interior. The reduced contact between the insertion element (especially the stabilizing element) and the insertion device or the implant beneficially achieves reduced friction and therefore easier introduction of the insertion device or the implant. Additionally the amount of material of the stabilizing element is enhanced which leads to an enhanced stiffness of the insertion element and therefore better push-ability of the insertion element.

Profiled circumferential segments with an enhanced stiffness are advantageously configured in the same way.

The sleeve may advantageously be extruded from at least two compatible materials at the distal or proximal end (when the sleeve is assembled), said materials forming the different circumferential segments of the sleeve. The primary material, that is to say the matrix, is additionally reinforced by a stabilizing element or by a plurality of stabilizing elements, which is/are incorporated during the extrusion process, thus providing the extruded sleeve with advantageous mechanical properties for the insertability of the sleeve and for the displacement properties of the sleeve along the insertion device. The expand ability of the diameter of the sleeve is provided by the properties of the at least two types of circumferential segments.

The medical implant, for example a self-expanding prosthesis such as a stent or a cardiac valve, has to be placed at a defined position and released from the insertion device. The sleeve stabilized by stabilizing elements can advantageously be slid, if necessary, from the proximal end of the insertion device over the outer shaft and in the direction of the implant at the distal end. The stabilizing element(s) allow(s) the sleeve to be slid over reliably. If an implant partially released from the insertion device at the distal end is positioned incorrectly or disadvantageously, the sleeve can be slid over the implant and the implant can be sheathed and repositioned, or can be gently removed completely again. It is particularly advantageous that the surface of the sleeve is smooth, so that the risk of catching is prevented. Sharp edges, points and slits are sheathed reliably by the sleeve.

In accordance with an advantageous embodiment, a friction-reducing coating can be provided on the sleeve. The handling of the insertion element and its use in an insertion device is thus facilitated and can be implemented more quickly and therefore in a less stressful manner for a patient. The coating may be a hydrophobic coating or a hydrophilic coating. The coating may be arranged on the inner side of the sleeve, on the outer side of the sleeve, or on the inner side and outer side of the sleeve. It can be hydrophilic on both sides or hydrophobic on both sides, or hydrophilic on the inner side and hydrophobic on the outer side, or hydrophobic on the inner side and hydrophilic on the outer side.

In accordance with an advantageous embodiment, one type of the circumferential segments may be embedded, in the form of a narrow strip extending in the longitudinal extension of the sleeve (i.e., in the direction of a sleeve axis), in a second type of circumferential segments used as a matrix. One strip may be provided, or two or more strips may be provided. The number of strips can be selected according to the materials used for the first and/or second circumferential segment and/or according to desired ductile properties of the sleeve. As required, it is also possible to select whether the material is arranged with greater or lesser resilience in the strips or in the matrix.

In accordance with an advantageous embodiment, the sleeve may have at least two circumferential segments of one type in the circumferential direction, which are each separated by a circumferential segment of the other type. This provides a particularly simple geometry of the sleeve when expanding the diameter.

In accordance with a further aspect of the invention, an insertion device for inserting a medical implant into a human or animal body is proposed, comprising at least one outer insertion element, which is resiliently expand able, at least in regions, when ready for use, wherein the outer insertion element is formed as a sheath, through which an outer shaft, which surrounds an inner shaft on which a medical implant for insertion into a human or animal body is arranged, can be inserted at a site of use. In this case, the outer insertion element comprises a sleeve, which, in the circumferential direction, has at least two (and in some embodiments three, four, five or more) types of circumferential segments of different resilience. Due to the ductility of the insertion element when the implant or a catheter comprising an implant is passed through, the opening in the body for insertion of the implant can be smaller than if a sheath of maximum necessary diameter were used.

In accordance with a further aspect of the invention, an insertion device for inserting a medical implant into a human or animal body is proposed, comprising at least one outer insertion element, wherein the outer insertion element is formed at least as a distal end of an outer shaft, which surrounds an inner shaft and which sheathes a medical implant arranged on the inner shaft, at least before or during insertion into a human or animal body, wherein the outer insertion element is resiliently expand able, at least in regions, when ready for use. In this case, the outer insertion element comprises a sleeve, which, in the circumferential direction, has at least two types of circumferential segments of different resilience. Due to its ductility, the insertion element, that is to say the outer shaft, can be slid over an incorrectly positioned implant, for example a self-expanding stent, so that said stent can be repositioned or safely removed again from the body. If the insertion element has a region in which at least one stabilizing element is arranged, at least in regions, in at least one of the circumferential segments along a longitudinal extension of the sleeve, the outer shaft can be slid back in a particularly simple and stable manner. Even if a repositioning of the implant were unsuccessful, the insertion element, which has been slid on again, surrounds the implant such that said implant causes fewer micro injuries in the vessels if the implant is removed again.

The insertion devices according to the various aspects of the invention may optionally also be combined, such that an advantageous insertion device is produced, of which the outer shaft has a distal end, which is resiliently expand able at least in regions, and which can be introduced through a resiliently expand able sheath. In this case, the sheath and at least the distal end of the outer shaft have at least one of the features of the above-described insertion element.

With all proposed insertion devices, the outer insertion element may advantageously have a region in which at least one stabilizing element is arranged, at least in regions, in at least one of the circumferential segments along a longitudinal extension of the sleeve. The insertion element is thus particularly stable.

Turning now to the figures, functionally like or like-acting elements are each denoted by like reference signs. The figures are schematic illustrations of the invention. They do not show specific parameters of the invention. Furthermore, the figures merely illustrate typical embodiments of the invention and are not intended to limit the invention to the illustrated embodiments.

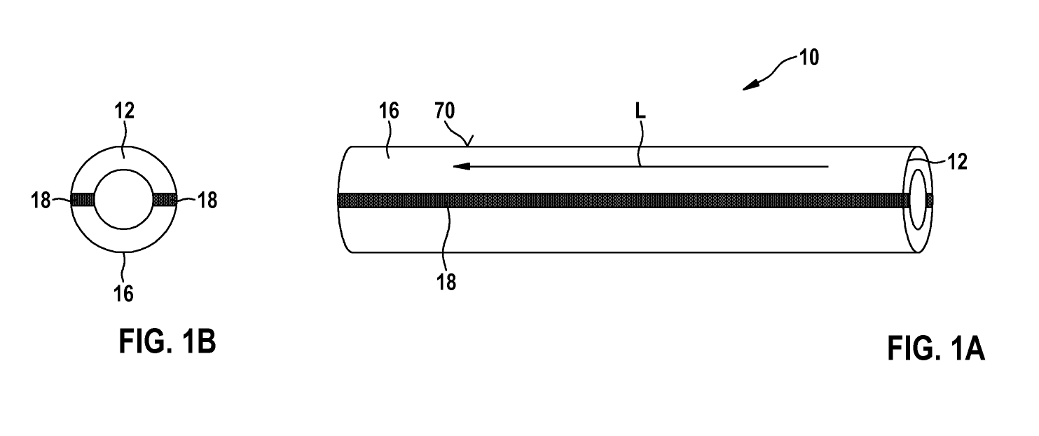

FIGS. 1A and 1B show a first exemplary embodiment of an insertion element 10 in a side view (FIG. 1A) and in cross section (FIG. 1B). The insertion element 10 may, in particular, be used as a sheath 80 for a catheter for example or may be a direct component of a catheter, for example an outer shaft of a catheter.

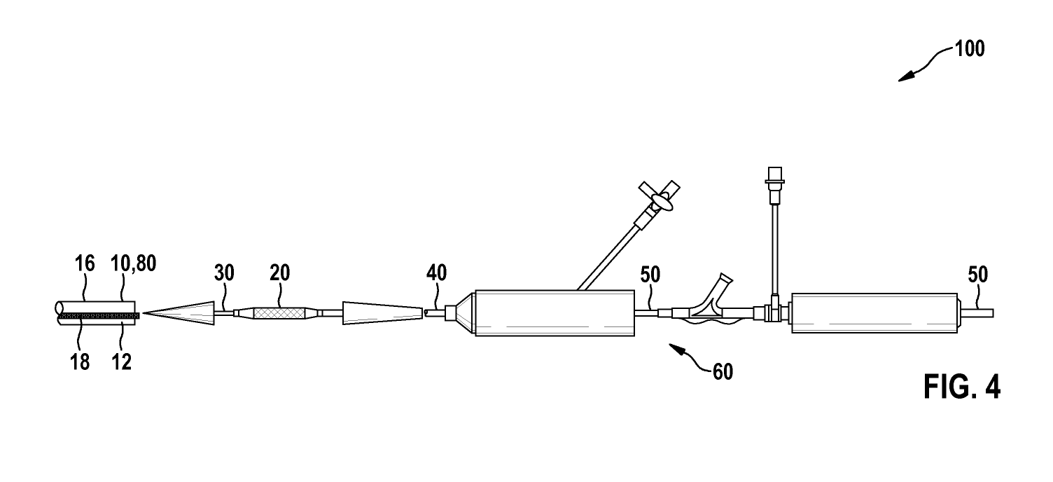

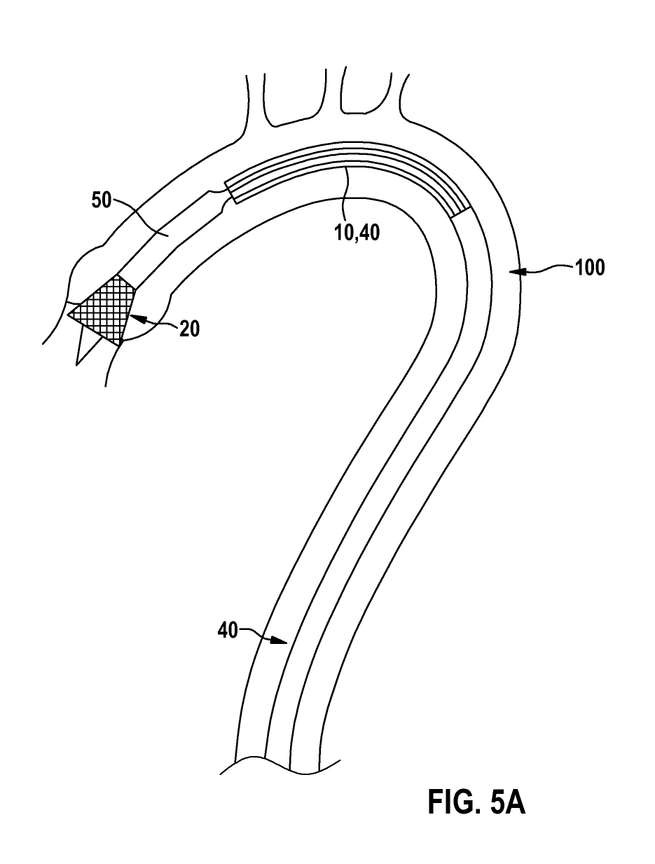

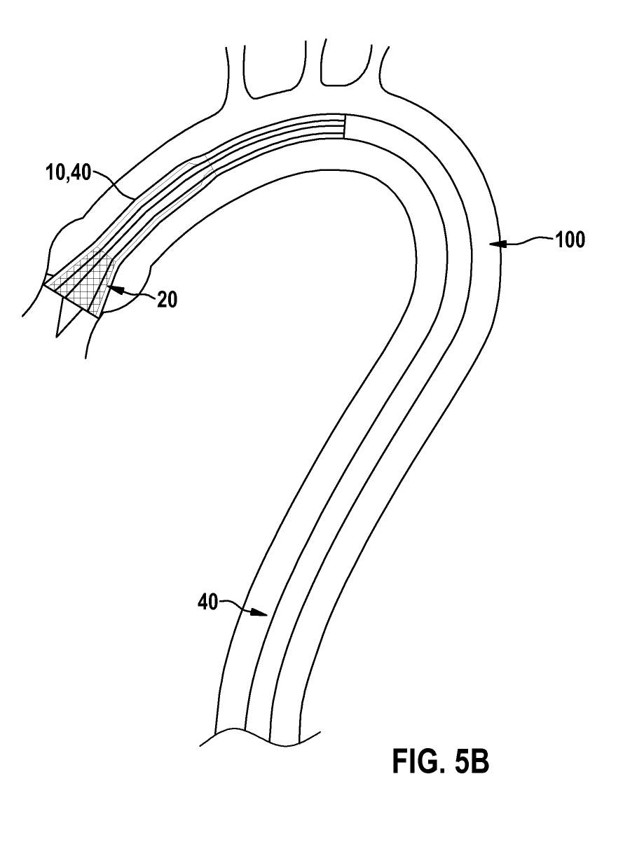

The insertion element 10 is designed to cooperate with an insertion device 100 (FIGS. 4, 5A and 5B) for inserting a medical implant 20, for example a self-expanding stent, into a human or animal body. The tubular sleeve 12 of the insertion element 10 has, in the circumferential direction, at least two circumferential segments 16, 18 of a first and a second type of different resilience, which are diametrically opposed for example. The second circumferential segment 18 is formed as a narrow strip along the longitudinal extension L of the insertion element 19.

The sleeve 12 has inner and outer wall surfaces, and as illustrated the thickness of the segments 16 and 18 in the radial direction are substantially constant so that the sleeve 12 has a uniform wall thickness. In other embodiments, radial direction of the segments 16 and 18 can vary to provide, for example, varying ductility or resistance to stretching. As illustrated, the circumferential area of the segment 16 is significantly greater than that of segment 18. Differences in area can depend on design features such as differences in elasticity of the segments 16 and 18 as well as desired stretching of the sleeve 12. In some embodiments, the circumferential area of segment 16 is a factor of at least 6 times greater than that of segment 18.

The first circumferential segment 16 is much stiffer than the second circumferential segment 18. The first, rigid circumferential element 16 is formed from polyamide PA 12 for example, and the second, resilient circumferential segment 18 is formed from polyamide elastomer.

The insertion element 10 may optionally have a friction-reducing coating 70, for example on the outer side of the sleeve 12. The coating 70 may have hydrophilic or hydrophobic properties.

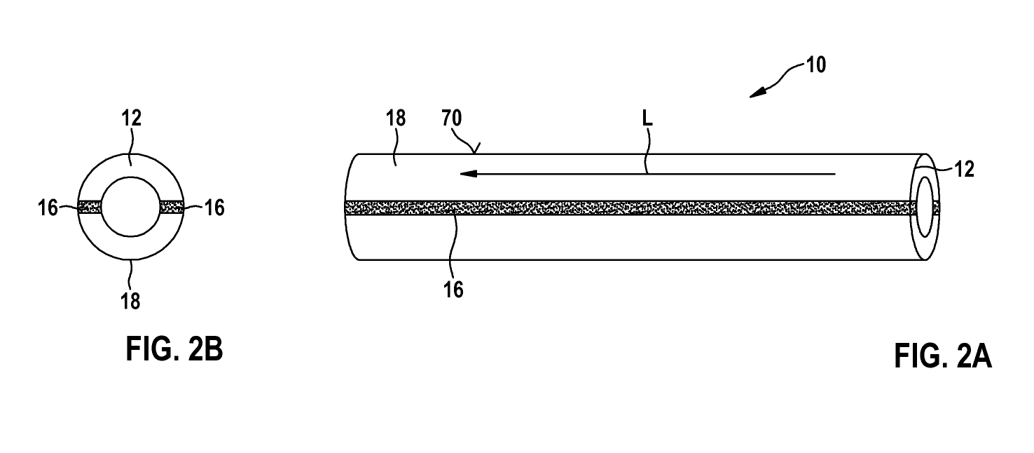

FIGS. 2A and 2B show a further exemplary embodiment of an insertion element 10 in a side view (FIG. 2A) and in cross section (FIG. 2B).

The insertion element 10 is designed to cooperate with an insertion device 100 (FIGS. 4, 5A and 5B) for inserting a medical implant 20 into a human or animal body. The tubular sleeve 12 of the insertion element 10 has, in the circumferential direction, at least two circumferential segments 16, 18 of different resilience, which are diametrically opposed for example. The second circumferential segment 18 is formed as a broad strip along the longitudinal extension L of the insertion element 10, in which the first circumferential segment 16 is embedded in the form of a narrow strip.

The first circumferential segment 16 is much stiffer than the second circumferential segment 18. The first circumferential element 16 is formed from polyester for example, and the second circumferential segment 18 is formed from polyester elastomer. As illustrated in FIGS. 1 and 2, segments 16 and 18 are substantially evenly spaced relative to one another about the perimeter of the sleeve 12. In other embodiments spacing is irregular as may be useful, for example, to concentrate the stretching ability of the sleeve 12 in a particular circumferential region. Adopting the reference of a clock face, for example, segments 18 could be placed at 12 o'clock, 2 o'clock, 4 o'clock and 6 o'clock to concentrate stretching of the sleeve in this half-perimeter as compared to the 6 o'clock to 12 o'clock half-perimeter.

The insertion element 10 may optionally have a friction-reducing coating 70, for example on the outer side of the sleeve 12. The coating 70 may have hydrophilic or hydrophobic properties.

The circumferential segments 16, 18 of the sleeve 12 shown in the exemplary embodiments are advantageously formed by materials that are compatible with one another, from which the sleeve 12 can be produced particularly easily by co-extrusion. The strip-shaped circumferential segments 16 or 18 are embedded in the half-shell-shaped circumferential segments 18 or 16, as in a matrix. The circumferential segments 16, 18 together form a covering jacket, which is closed in the circumferential direction and which can be expanded reversibly when the implant 20 (FIG. 1) passes the respective point within the insertion element 10.

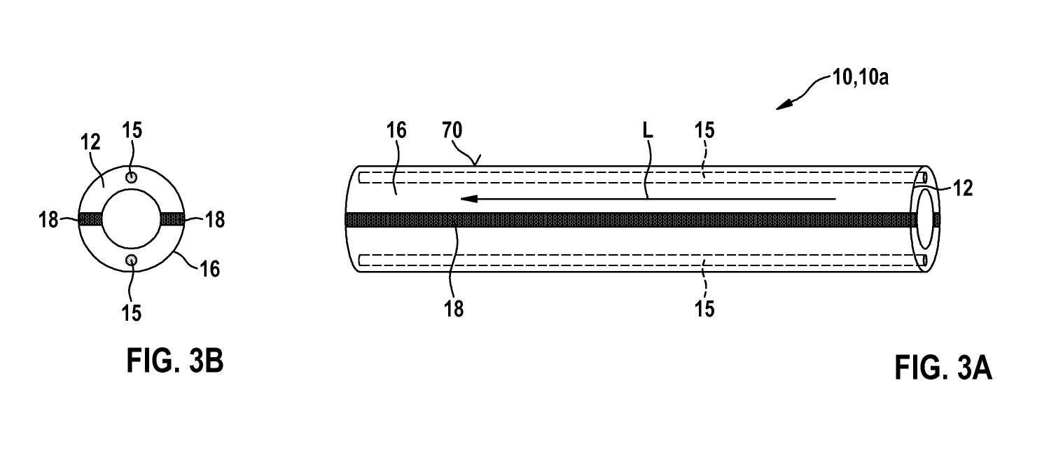

FIGS. 3A and 3B show a further exemplary embodiment of an insertion element 10 in side view (FIG. 3A) and in cross section (FIG. 3B). The insertion element 10 has at least one stabilizing element 15, for example a wire, in particular a metal wire, at least in regions (region 10a), in at least one of the circumferential segments 16, preferably in the region of lesser ductility, along a longitudinal extension L of the sleeve 12. The stabilizing element 15 boosts the mechanical stability of the insertion element 10. Stabilizing elements 15 can, in principle, be provided in one or more or all circumferential segments 16, 18, even in different types of circumferential segments 16, 18 of different resilience.

Advantageous materials for the rigid (less resilient) circumferential segments 16 according to FIGS. 1, 2 and 3 are PEEK, PI, PE, PP, PC, preferably polyamides such as PA12 (for example Grilamid L25), or polyester such as PET, and polyamide elastomers or other materials with Shore D hardness >60, preferably with shore D hardness >65, or polyester elastomers with shore D hardness >60, preferably with shore D hardness >65. Materials for the resilient circumferential segments 18 according to FIGS. 1, 2 and 3 are TPE-Os, TPE-Us, preferably polyamide elastomers (TPE-As) or other materials with shore D hardness <70, preferably with shore D hardness <65, or polyester elastomers (TPE-Es) with shore D hardness <70, preferably with shore D hardness <65. In this case, the resilient differences between the circumferential segments 16 and 18 are preferably rather pronounced.

To illustrate the invention, FIG. 4 shows a side view of a first exemplary embodiment of an insertion device 100, which in this case comprises a catheter for example, with which a self-expanding stent is to be inserted. The insertion device 100 comprises inner and outer insertion elements in the form of an inner shaft 50 and an outer shaft 40, which surrounds said inner shaft and at the proximal end of which a conventional handling device 60, not described in greater detail, is arranged, comprising different connection pieces, for example for flushing the lumen of, or between, the inner shaft 50 and outer shaft 40 and for releasing the implant 20. An implant 20 with a conventional guide device 30 is arranged on the inner shaft 50 between the opposed distal ends.

To insert the catheter or the assembly of the outer shaft 40, the inner shaft 50 surrounded by the outer shaft 40, and the implant 20 with guide device 30 arranged on the inner shaft 50 into the human or animal body, said catheter or assembly is slid through an insertion element 10 formed as a sheath 80, with the implant 20 or the guide device 30 at the front. To insert the catheter, a flexible sheath 80 is first introduced into the blood vessel for example, through which the catheter can slide into the blood vessel. As described in the exemplary embodiments discussed above, the insertion element 10 formed as a sheath 80 comprises a sleeve 12 with a circumferential segment 16 of a first type and a circumferential segment 18 of a second type, wherein the two types differ in terms of their resilience. The circumferential segment 18 of the second type is formed as a narrow strip along the longitudinal extension of the tubular insertion element 10, said strip being embedded in the matrix-like circumferential segment 16 of the first type.

To illustrate the invention, FIGS. 5A and 5B show a side view of a further exemplary embodiment of an insertion device 100, in which an insertion element 10 is provided as a distal end of an outer shaft 40. As described in the exemplary embodiments discussed above, the insertion element 10 comprises a sleeve 12 with a circumferential segment 16 of a first type and a circumferential segment 18 of a second type, wherein the two types differ in terms of their resilience. The circumferential segment 18 of the second type is formed as a narrow strip along the longitudinal extension of the tubular insertion element 10, said strip being embedded in the matrix-like circumferential segment 16 of the first type. The outer shaft 40 can be formed completely, or merely in regions, as an insertion element 10, in particular at the distal end of the outer shaft 40 in the region that sheathes the implant 20.

In this case, a stabilizing element 15 as described in FIGS. 3A and 3B can particularly advantageously be provided in the sleeve 12. For example, a region of the sleeve 12 that is not additionally stabilized can adjoin the region of the sleeve 12 stabilized in this manner. An insertion situation of the insertion device 100 with a partly released implant 20 in the form of a self-expanding stent at the distal end of the insertion device 100 is illustrated in a side view in FIG. 5A.

The insertion situation of an insertion device 100 with an implant 20 re-sheathed with the insertion element 10 slid over is illustrated in a side view in FIG. 5B. The insertion element 10 may extend over the entire axial length of the insertion device 100 or the outer shaft 40, wherein, at the distal end thereof, a region 10a may advantageously be provided with one or more stabilizing elements 15.

So as to reposition the partly released implant 20 for example, the insertion element 10 may be slid over from the proximal end of the insertion device 100 in the distal direction, until the stabilized region 10a of the insertion element 10 has been slid at the distal end of the outer shaft 40 over the partly released implant 20 and sheathes it again completely. If necessary, the implant 20 can also be gently removed again in this state from the body, since the insertion element 10 sheathes sharp edges or pointed parts of the implant.

The insertion element 10 according to the invention may advantageously be used as a sheath 80 so as to insert the insertion device 100 into a human or animal body, and additionally or alternatively as an outer shaft 40 or part of the outer shaft 40 to “re-sheath” a partly released implant 20.

Due to the use of suitable materials for the circumferential segments 16, 18, the more ductile of the circumferential segments 16, 18 may advantageously have an elongation that is up to four times greater for example than the less ductile circumferential segments 16, 18.

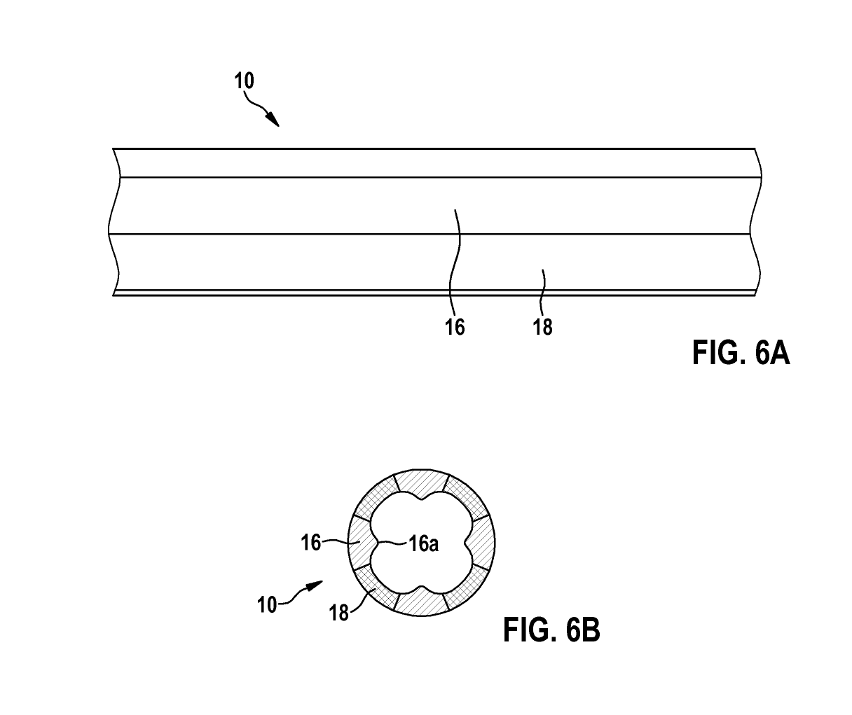

FIGS. 6A and 6B show a further example embodiment of an insertion element 10 in a side view (FIG. 6A) and in cross section (FIG. 6B). The insertion element 10 may, in particular, be used as a sheath 80 for a catheter.

The insertion element 10 comprises at least two different circumferential segments 16, 18 of different resilience, which are diametrically opposed and arranged adjacent to one another about an interior circumference of the element 10. The circumferential segments 16 have an enhanced stiffness and are made of molded metal strips. The circumferential segments 18 have an enhanced resilience and are made of polyamide elastomer. The metal strips 16 are embedded in the polyamide elastomer segments 18.

As shown in FIG. 6B the circumferential metal strip segments 16 are molded to result in an interior facing side having a tip 16a pointed to the inside of the insertion element 10. This interior surface and tip 16a extend further into the element interior 10 than does a corresponding surface of the elastomer segments 18, and the tip 16a has a generally tapered profile when viewed along the direction of a major axis of the element 10 as shown in FIG. 6B. In case the insertion element 10 is used as an introducer sheath 80 for a catheter, the contact area between the insertion element 10 and the introduced catheter is thereby minimized (i.e., the catheter will contact and be held in place through engagement with the tips 16a only). Other shapes in addition to the tips 16a are used in other embodiments, with the embodiments sharing the feature that a reduced area engaging surface is achieved. Additionally the stiffness of the whole insertion element 10 is enhanced by the increased amount of material in the circumferential segments 16 as compared to the segments 18.

The insertion element 10 shown in FIGS. 6A, 6B could be also used as distal part of the outer shaft 40 of an insertion device 100 as shown in FIGS. 5A, 5B.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. As an example, embodiments of the invention are not limited to only two different segments, or to equally distributed segments. Three or more segments can be provided, and they may be unequally distributed about the circumference of the interior. Therefore, it is the intent to cover all such modifications and alternate embodiments as may come within the true scope of this invention.

What is claimed is:

1. An insertion element, which is designed to cooperate with an insertion device for inserting a medical implant into a human or animal body, comprising a sleeve, which, in the circumferential direction, has at least two circumferential segments of different resilience.

2. The insertion element as claimed in claim 1, characterized in that one of the circumferential segments is much stiffer in the circumferential direction than the other of the circumferential segments.

3. The insertion element as claimed in claim 2, wherein the sleeve has a tubular shape, wherein the at least two circumferential segments extend along a longitudinal extension (L) of the insertion element.

4. The insertion element as claimed in claim 1, characterized in that one of the circumferential segments comprises thermoplastic material and the second comprises a metal.

5. The insertion element as claimed in claim 4, characterized in that one of the circumferential segments comprises one or more of a polyamide and a polyester.

6. The insertion element as claimed in claim 5, characterized in that the other of the at least two circumferential segments comprises a one or more of a polyamide elastomer and a polyester elastomer.

7. The insertion element as claimed in claim 1, and further comprising at least one stabilizing element arranged, at least in regions, in at least one of the at least two circumferential segments along a longitudinal extension of the sleeve.

8. The insertion element as claimed in claim 1 and further comprising a friction-reducing coating, and wherein one of the segments has an interior surface tip having a tapered profile that extends farther into the element interior than does the interior surface of the other of the segments to define a contact area between the insertion element and a catheter that is introduced into the insertion element interior.

9. The insertion element as claimed in claim 1, characterized in that at least one of the circumferential segments is arranged in a matrix of the second of the circumferential segments in the form of a narrow strip in a matrix.

10. The insertion element as claimed in claim 1, characterized in that the at least two circumferential segments comprise at least two circumferential segments of a first resilience in the circumferential direction, which are each separated by a circumferential segment of a second resilience.

11. The insertion element as claimed in claim 1, characterized in that it has a resiliently expand able diameter.

12. An insertion element as claimed in claim 1, and further comprising a device for inserting a medical implant into a human or animal body, the insertion element arranged as an outer element on the device wherein the insertion element is formed at least as a distal end of an outer shaft of the device, which surrounds an inner shaft and which sheathes a medical implant arranged on the inner shaft, at least before or during insertion into a human or animal body, wherein the outer insertion element is resiliently expand able, at least in regions, when ready for use.

13. The insertion element as claimed in claim 13 for inserting a medical implant into a human or animal body, wherein the outer insertion element is formed as a sheath, through which an outer shaft, which surrounds an inner shaft on which a medical implant for insertion into a human or animal body is arranged, can be inserted at a site of use.

14. The insertion element as claimed in claim 13, characterized in that the outer insertion element has a region in which at least one stabilizing element is arranged, at least in regions, in at least one of the circumferential segments along a longitudinal extension of the sleeve.

15. The insertion element as claimed in claim 1 wherein a first of the two circumferential segments has a modulus of elasticity that is at least twice the modulus of elasticity of a second of the circumferential segments.

16. The insertion element as claimed in claim 1 wherein each of the at two circumferential segments have a circumferential width and a length in the axial direction of the sleeve, and wherein the circumferential width of at least one of the at least two circumferential segments varies along its length to result in a length variable ductility of the insertion element.

17. The insertion element as claimed in claim 1 and further comprising a stabilizing element embedded within a first of the at least two circumferential segments, the first circumferential segment having a circumferential area that is greater than any of the other circumferential segments.

18. The insertion element as claimed in claim 1 and further comprising a first sleeve region proximate to a first sleeve end, and a second sleeve region proximate to a second distal sleeve end, a stabilizing element embedded in one of the circumferential segments in the first sleeve region but not in the second sleeve region.

19. The insertion element as claimed in claim 1, wherein the sleeve has a tubular shape, wherein the at least two circumferential segments extend along a longitudinal extension (L) of the insertion element, one of the two segments having an internal surface that extends further into an insertion element interior than does the internal surface of the other of the two segments.

20. An insertion element configured to cooperate with an insertion device for inserting a medical implant into a human or animal body, comprising:
a tubular sleeve having an axial length, the sleeve defined by a plurality of first circumferential segments that are separated by a plurality of second circumferential segments, each of the first and second segments extending over the entire sleeve length, the first circumferential segments having a modulus of elasticity that is at least twice the modulus of elasticity of the second circumferential segments, the second circumferential segments having a circumferential area that is at least six times that of the circumferential area of the first circumferential segments;
a reinforcing element embedded in the first circumferential segment and extending in a longitudinal direction of the tubular sleeve; and,
a friction reducing coating on at least one surface of the tubular sleeve.
